(19) United States
(12) Patent Application Publication
(10) Pub. No.: US 2025/0177145 A1
(43) Pub. Date: Jun. 5, 2025

(54) METHOD OF MANUFACTURING A MEDICAL DEVICE USING 3D PRINTING AND ELECTROSPINNING

(71) Applicant: INSTITUTO DISTRITAL DE CIENCIA BIOTECNOLOGIA E INNOVACIÓN EN SALUD - IDCBIS, Bogotá (CO)

(72) Inventors: William Antonio CÁRDENAS AGUAZCO, Bogotá (CO); Adriana Lorena LARA BERTRAND, Bogotá (CO); Ingrid Zulay SILVA COTE, Bogotá (CO)

(21) Appl. No.: 18/844,472

(22) PCT Filed: Mar. 7, 2023
(86) PCT No.: PCT/IB2023/052110

§ 371 Date: Sep. 6, 2024

(30) Foreign Application Priority Data
Mar. 7, 2022 (CO) NC2022/0002677

(51) Int. Cl. A61F 2/28; A61L 27/12; A61L 27/32

(52) U.S. Cl. CPC A61F 2/28; A61L 27/12; A61L 27/32

(57) ABSTRACT

A method of manufacturing a medical device made of polylactic acid (PLA) and hydroxyapatite (HA) aimed at the repair of fractures or considerable bone injuries, using 3D printing and electrospinning techniques.

### FIELD OF THE INVENTION

The present invention relates to a method of manufacturing a medical device by 3D printing and electrospinning useful in bone repair.

The invention has applicability in the pharmaceutical industry, in particular, in bone tissue repair processes.

### PRIOR ART

Conventional treatments used in considerable bone losses do not achieve tissue regeneration, which causes multiple surgical interventions that often result in the reduction of the quality of life of patients and increase in costs in health services. Likewise, treatments do not guarantee a complete repair of the tissue and patients can be left with significant sequelae.

Such treatments used for the repair of considerable bone defects are based on the use of autografts, allografts or methacrylate cements. Autografts are the main alternative; however, their use is limited due to the low amount of material available. On the other hand, methacrylate cement is used, however, it does not have a porous architecture and has no defined shape, therefore, the orthopedist must mold the material during the surgical process and adapt it to the missing bone tissue. In addition, methacrylate does not have biodegradation processes and can present deficiencies in the mechanical field, therefore it cannot be used in all bone tissues.

In view of the complexity demanded by bone repair treatments, the application of a highly versatile technology that is three-dimensional (3D) printing by additive manufacturing has been explored in recent years. 3D printing provides greater flexibility in the control of the microarchitecture of three-dimensional structures (see B. G. Compton, J. A. Lewis; 3D-Printing of Lightweight Cellular Composites, Adv. Mater. 26 (2014) 5930-5935). The great interest in this technique lies in its versatility and easy use since it allows the construction of three-dimensional objects through the layer-by-layer deposition of a material following a printing pattern which is previously configured in a Gcode file (see T. D. Ngo, A. Kashani, G. Imbalzano, K. T. Q. Q. Nguyen, D. Hui, Additive manufacturing (3D printing); A review of materials, methods, applications and challenges, Compos. Part B Eng. 143 (2018) 172-196). This allows customized, macro/micro porous structures with tuned mechanical properties to be fabricated from polymers, ceramics, metals, or composites (see, e.g., S. C. Ligon et al.; Polymers for 3D Printing and Customized Additive manufacturing, Chem. Rev. 117 (2017) 10212-10290). Due to the morphological and structural features obtained by 3D printing, medical implants, tools, instruments and devices have been designed and developed, with high quality standards in a shorter time and costs (see for example Y. W. D. Tay et al.; 3D printing trends in building and construction industry: a review, Virtual Phys. Prototyp. 12(2017) 261-276).

Likewise, we also have the electrospinning method that allows obtaining fibers of micro and/or submicron diameters. This feature gives high traction, surface area and porosity, among others, to two-dimensional membranes. The fibers obtained by electrospinning can be polymeric or from combinations of organic and inorganic materials, which have enormous potential to significantly improve processes such as filtration, release, applicability in textiles, among others; as well as for the development of new applications in tissue engineering (see for example S. Ramakrishna et al.; Δn introduction to electrospinning and nanofibers. (2005) 1-382).

In this regard, the state of the art shows various developments in this regard. For example, US publication US2016067375A1 discloses methods for creating 3D bio-inspired tissue engineering scaffolds with excellent interfacial mechanical properties and biocompatibility, and products created with such methods. Wherein a combination of nanomaterials, nano/microfabrication methods and 3D printing can be employed to create structures that promote tissue reconstruction and/or production. European Publication EP3003224A1 discloses synthetic bone graft materials or tissue engineered scaffolds with desired structural and biological properties (e.g., well-controlled macroporosities, spatially defined biological microenvironment, good handling features, self-anchoring ability, and shape memory properties) and methods for their application in vivo. Also, European publication EP3316807A1 discloses a bone anchor having a bone abutment surface adapted for congruent fastening to a bone and methods for the production of said bone anchor. Wherein the manufacturing methods generally comprise the mapping of the shape of the bone to which the bone anchor is to be applied, commonly performed by an imaging technique. The manufacturing methods further include any suitable process used to manufacture a three-dimensional object, said process generally including additive or subtractive manufacturing methods. European publication EP3829663A1 discloses a 3D printed surgically implantable tissue engineering scaffold for promoting bone, vascular and/or cartilage regeneration in osteochondral regions and a method of manufacturing the 3D printed surgically implantable tissue engineering scaffold. On the other hand, US publication U.S. Ser. No. 11/168,412B2 discloses a modified electrospinning method of manufacturing uniformly shaped and porous nanofibrous scaffolds that can be used in various applications, useful in guided bone regeneration.

However, the prior art does not provide the combination of 3D printing and electrospinning for the manufacture of a medical device applied in bone repair can be of great applicability at the clinical level. For the development of this type of device, the materials to be used, such as biodegradable synthetic polymers, must be evaluated since their degradation products do not affect the integrity of the patient. Among the most used polymers are polylactic acid (PLA), polycaprolactone, polyglycolides (PLG), poly(lactide-co-glycolide) copolymers (PLGA) and polyalkylcyanoacrylates, since it is biocompatible, its physicochemical features give it a bond between layers and the pieces maintain their shape at body temperature. In addition, this material has been applied in research related to tissue engineering through the manufacture of three-dimensional structures, known as scaffolds, which stabilize the area to be treated while carrying out the natural process of tissue repair.

Due to the foregoing, the present invention provides a method of manufacturing a device for providing bone repair in clinical application by the 3D printing and electrospinning methods, using biomaterials approved by the referring health authorities, such as the United States Food and Drug Administration (FDA). The method of the invention allows to elaborate a device having a three-dimensional structure with macroporous architecture that guarantees the transit of cells, nutrients and factors associated with bone repair processes, favoring the formation of new bone in the critical bone defect. In addition, it has a bioactive coating that limits the invasion of adjacent tissue and contributes to cell differentiation processes.

The invention allows to establish the design and modeling process of the devices from various diagnostic images, combine two techniques such as 3D printing (also called additive manufacturing) and electrospinning in the manufacture of scaffolds, parameterize the factors involved in the manufacturing process either in 3D printing or in electrospinning, introduce a channel into the device that allows the transit of bone marrow in the affected tissue and generate a chemically activated polymeric coating that induces bone repair

### DESCRIPTION OF THE DRAWINGS

FIG. 1 shows the CAD model of the scaffold for the first in vitro studies.

FIG. 2 shows the compression specimens according to the standard ASTM D695

FIG. 3 shows comparison of the Young's moduli obtained with the different infill patterns of the different structures of the compression specimens.

FIG. 4 shows a comparison between infill densities and 60%, 50%, 40%, 30% and 20%.

FIG. 5 shows the design, modeling and incorporation of the medullary canal into the three-dimensional structure.

FIG. 6 shows the evaluation of the volumetric capacity of the 3D PLA scaffold with culture medium.

FIG. 7 shows the images obtained by the fluorescence microscope of the viability assay (“live/dead”) of the CEM-GW seeded on the PLA 3D scaffolds.

FIG. 8 shows the fluorescence microscope images corresponding to the viability assay (“live/dead”) performed on the coated scaffolds.

FIG. 9 shows a cell viability evaluation of 3D PLA scaffolds and electrospun in GENbag with CO₂ bag.

FIG. 10 shows 3D scaffolds, electrospun and control after 4 hours in GENbag at room temperature and with CO₂ generator.

FIG. 11 shows the viability assay (“live/dead”) of the cells seeded in 3D Quyne and in the control after being subjected to room temperature and variable CO2 atmosphere for 4 hours.

FIG. 12 shows the design and modeling of the 3D device adapted for critical bone injury in rabbit femur.

FIG. 13 shows an impression simulation of the device designed for a critical bone defect in lagomorphic biomodel femur.

FIG. 14 shows CAD design, Slic3r programming, and printed container employed in 3D PLA scaffold cell seeding.

FIG. 15 shows the “3D Quyne” device prepared for assaying in biomodels.

FIG. 16 shows the 3D scaffold cell culture assay with PLA container.

FIG. 17 shows images of the viability (“live/dead”) of (A) CEM-GW grown in the 3D Quyne device and (B) control CEM-GW seeded in culture dish subjected to variable CO₂ atmosphere and room temperature for 4 hours.

FIG. 18 shows the electrospun membrane biomodel.

FIG. 19 shows the Positive Control: Rabbit with autograft.

FIG. 20 shows the X-rays of the biomodels with coated 3D PLA scaffold.

FIG. 21 shows the radiographs of the negative control biomodel.

FIG. 22 shows the Biomodel radiograph with 3D Quyne device.

FIG. 23 shows Biomodel X-ray with functionalized 3D Quyne device.

FIG. 24 shows the graphical representation of the functioning of the spinal canal, vascularization and porosity of the 3D Quyne device.

FIG. 25 shows the visualization of the 3D Quyne device extracted from the bone tissue and the visualization of possible calcifications and cells within the pores of the device.

FIG. 26 shows the visualization of calcifications, cells and blood vessels found in the 3D Quyne device upon completion of the in vivo assay.

FIG. 27 shows the implant simulation of the 3D Quyne device in the femur.

### DETAILED DESCRIPTION OF THE INVENTION

The present invention relates to a method of manufacturing a medical device made of a biodegradable polymer selected from the group consisting of polylactic acid (PLA), polycaprolactone, polyglycolides (PLG), poly(lactide-co-glycolide) (PLGA) copolymers and polyalkylcyanoacrylates, together with hydroxyapatite (HA), wherein the device is aimed at the repair of considerable bone fractures or injuries.

Biodegradable polymers are particles that show lower systemic toxicity and are more biocompatible with tissues, as they are usually degraded into oligomers and monomers, which are then metabolized and eliminated from the body by normal pathways. Polylactic acid (PLA), polycaprolactone, polyglycolides (PLG), poly(lactide-co-glycolide) copolymers (PLGA), and polyalkylcyanoacrylates are polymers that have been approved by the U.S. Food and Drug Administration (FDA) as well as the European Medicines Agency (EMA) for pharmaceutical application.

For example, PLA is a representative and widely used bio-based biodegradable polymer that can be produced from renewable resources, including corn and potato starch, beet sugar, and sugar cane, among others. Since it is a bioabsorbable material, it can be used in surgical procedures either in the form of scaffolds, fastening devices such as screws, nails, washers, among others, to favor the recovery of thoracic, hand, leg, finger and toe fractures. It can also be used in ligament reconstruction procedures; soft and hard tissue fastening; alignment of bone and osteochondral fragments; meniscus repair; and hyaline cartilage fastening. Among the striking features of PLA are its mechanical properties similar to trabecular bone tissue and its degradation process, which begins with the hydrolysis of the polymer chain and ends with the obtaining of CO₂ and H₂O, which are incorporated into the Krebs cycle.

On the other hand, hydroxyapatite (HA) is an isomorphic compound that normally occurs in calcium phosphate. This material has a hexagonal structure, with a crystal lattice composed of a compact set of phosphate groups (PO₄). There are various ways of obtaining hydroxyapatite; however, chemical synthesis methods show a higher yield. Said process comprises the synthesis from different starting compounds, such as calcium oxide (CaO), calcium hydroxide (Ca(OH)₂), phosphoric acid (H₃PO₄), sodium triphosphate (Na₅P₃O₁₀), among others. This compound has a Ca/P molar ratio ranging from 1.50 to 1.67, its molecular formula is Ca₅(PO₄)₃OH and is usually used in bone regeneration processes due to its excellent bioactivity, biocompatibility and contribution to cell differentiation.

The method of manufacturing the device comprising a design step starting from diagnostic images such as 3D radiographs and axial tomography, which are displayed in a DICOM format program for diagnostic images. The DICOM format program allows to observe the compromised bone tissue and the area of interest is delimited, which facilitates image cleaning and modeling in the computer-aided design (CAD) program. In the CAD, the dimensions of the implant are also established, the channel inside the device is designed and the external edges of the device are modeled according to the area of the injury. There, the diameter of the inner channel is configured, which corresponds to half the thickness of the compromised bone tissue.

Once the design stage is finished, we proceed with the 3D printing stage, where the model is imported into the Slic3r format 3D printing lamination software. In the 3D printing lamination software, the device dimensions are corroborated and the printing parameters are set, such as the nozzle temperature ranging from about 200° C. to about 215° C., the printing platform temperature ranging from about 30° C. to about 70° C., the layer height ranging from about 0.1 mm to about 0.2 mm, the wall thickness or width of the printing path ranging from about 0.2 mm to about 0.4 mm, and the printing speed being about 70 mm/s. As for the internal architecture of the device, this is done by means of a gyroid infill pattern and a print density of about 40% or a distance between lines that varies between 0.8 mm and 1.1 mm. The internal architecture confers on the device an interconnected macroporous three-dimensional structure and pore sizes ranging from about 200 micrometers to about 800 micrometers, said macropores allowing the diffusion and transport of nutrients through the internal architecture of the device. The device is then printed on a fused deposition modeling (FDM) machine layer by layer on PLA, polycaprolactone, PLG, PLGA, or polyalkylcyanoacrylates.

After obtaining the device by 3D printing, the device is coated with a polymeric membrane comprising polylactic acid (PLA) and hydroxyapatite. Said coating is obtained by electrospinning employing between about 7% and about 20% PLA, between about 1% and about 5% HA and 2,2,2-trifluoroethanol as solvent. The solution is subjected to about 37° C. and agitation ranging from about 400 rpm to about 600 rpm for at least 5 hours. The coating is obtained by electrospinning between about 0.2 mL and about 0.5 mL of polymer solution directly onto the 3D device, which is manually rotated using a voltage ranging between about 10 kV and about 18 kV, a distance between about 10 cm and about 18 cm, and an injection rate between about 0.6 mL/h and about 3 mL/h. In a preferred embodiment the coating is obtained by electrospinning about 0.3 of polymer solution directly onto the 3D device, which is manually rotated using a voltage of about 15 kV, a distance of about 15 cm and an injection rate of about 2 mL/h. The polymeric membrane has the function of promoting bone differentiation processes and in turn preventing the invasion of tissues adjacent to the site of injury in order to reduce the risk of delays in bone repair processes in this type of injury.

Subsequently, the coated device is taken to a convection drying oven, which is at approximately 37° C. In the drying oven it remains for approximately 48 hours, which is the time that guarantees the removal of residual solvent from the polymeric membrane. Finally, the device is subjected to gamma radiation sterilization with a range ranging from about 20 kGy to about 25 kGy.

In another embodiment of the invention is provided a device obtained by the above-described method comprising polylactic acid (PLA), polycaprolactone, PLG, PLGA or polyalkylcyanoacrylates and being coated by a polymeric membrane comprising between about 7% and about 20% PLA and between about 1% and about 5% HA.

The device of the invention can be functionalized with growth factors such as VEGF, FGF, PDGF, BMP2, BMP4, BMP7, among others. In addition, the device can be used as a scaffold to generate a tissue construct by culturing cells of mesodermal origin.

The method of manufacturing the device according to the invention and the device itself that allow to provide a customized medical device designed from diagnostic images, providing a model according to the bone defect to be treated and avoiding the molding of materials such as methacrylates or autologous bone tissue. When the device according to the invention is implanted in the bone defect, the channel of the device allows the transit of blood components from the bone marrow to the tissue, this promotes vascularization, recruitment and cell migration. Said device channel can be modified in the design software according to the area of interest, either for diaphysis or epiphysis injuries. Additionally, the macroporous internal architecture mimics natural trabecular bone tissue and facilitates nutrient transport, cell migration, and vascularization into neoformed bone tissue. As for the coating, its chemical composition gives it osteoinductive and osteoconductive properties that benefit bone repair and formation.

### Example 1. Design, Development and In Vitro Evaluation of the 3D Quyne Device

### Design and Development

The 3D Quyne device was developed from 3D printing, since this technique allows to manufacture, model and build mass or custom structures with controlled pore sizes and shape, by casting the polymeric material.

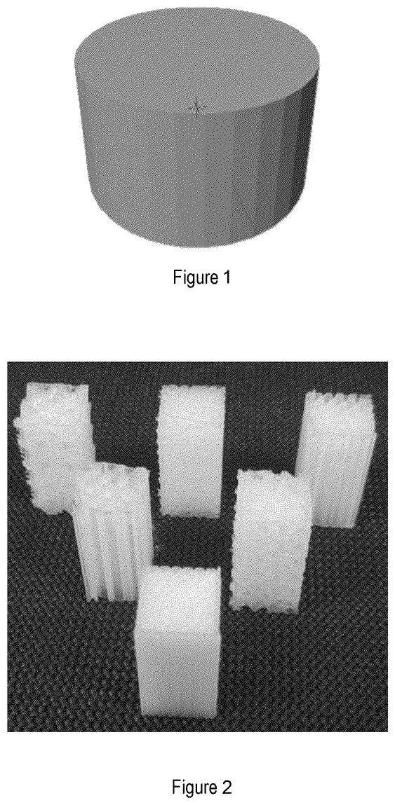

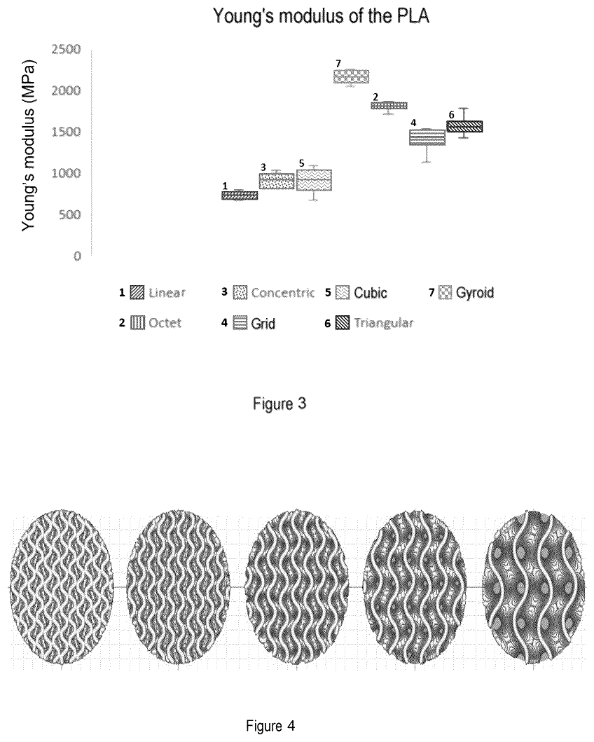

For in vitro assays a cylindrical scaffold developed in specialized CAD software was designed having a diameter of 10 mm and 5 mm high (see FIG. 1). Subsequently, the file is exported in the Standard Triangular Language (STL) format to continue with the configuration in the cutting program, where the variables (percentage of infill, infill pattern, temperature, printing speed, among others) that were previously parameterized for 3D printing with PLA filament are established. In order to find the infill that presents the best mechanical resistance to compression, a comparative analysis of different patterns (linear, concentric, cubic, gyroid, octet, grid and triangular) was carried out. For this purpose, compression specimens were manufactured with 100% infill and with dimensions according to ASTM D695 (See FIG. 2). In this assay it was possible to determine the Young's modulus values of the different structures of the compression specimens (see FIG. 3).

As observed in FIG. 3, the specimens with gyroid infill show a higher Young's modulus compared to the infill patterns reported in 3D printed devices. When choosing the gyroid pattern, the in vitro evaluation of the three-dimensional structure is continued. For this purpose, printing tests were carried out by varying the infill density of the device. In laboratory assays, it was found that a infill density of 40% is optimal, since pore sizes between 200 μm and 800 μm are obtained and their interconnection within the structure, which allows the transit of cells, growth factors and other cellular components involved in vascularization and osteogenesis processes. The following describes the parameters established for the manufacture of PLA-printed devices aimed at the repair of critical bone defects.

TABLE 1
Printing Parameters

| | |
|---|---|
| Infill: 40% | Platform temperature: 70° C. |
| Infill pattern: Gyroid | Bone position: vertical |
| Material: Polylactic acid (PLA) | Ventilation: 10% |
| Printing Speed: 50% | Support: 0 |
| Nozzle temperature: 205° C. | Scale: 1:1 |

A comparison between 40% and 60% fill density from the Cura software is presented in FIG. 4.

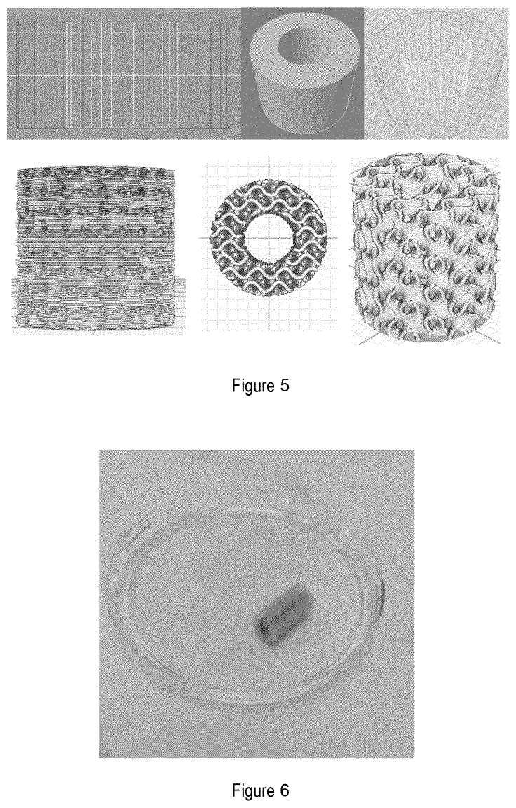

When defining the structural parameters that make up the device, the absence of a channel was noticed that allows the transit of bone marrow components, which is why a channel was incorporated in the center of the device with a diameter corresponding to half of the structure. Therefore, structures 10 mm high, 10 mm in diameter and a 5 mm diameter channel were printed in the center of the device, as shown in FIG. 5.

### In Vitro Assays of 3D Printed Device

After printing, the printed structures were brought to an in vitro evaluation under controlled conditions in order to determine the properties and behavior of both the material (PLA) and the Wharton's gelatin mesenchymal stromal cells (CEM-GW) seeded on these matrices. Given the hydrophobic nature of the polymer, the volume of culture medium that can be kept contained inside a scaffold of 2 cm high, 1 cm in diameter and medullary canal of 5 mm in diameter was estimated. Culture medium was deposited, its quantity gradually increasing through the channel. When reaching a volume of 500 μL, there was an overflow of more than half of the medium towards the bottom of the plate, reducing the wetting of the scaffold and therefore directly affecting cell viability in the structure. In view of the foregoing, it was established that 500 μL is the maximum volume that can be added to a structure with these features. The assay described above can be seen in FIG. 6.

In light of the above, a pilot trial was continued in which the evaluation of adhesion and cell viability of CEM-GW was carried out. These cells were selected due to their immunomodulation features and ability to differentiate into different tissues, including bone tissue.

Returning to the assay, the cell density calculation was made from the inventors' previous results. There, 2.5×10⁴ cells are used in a scaffold with a volume of 23.562 mm³ (10 mm in diameter and 0.075 mm in height). Given the above, the volume of the scaffolds is calculated through the following equation:

Cylinder volume=πr²h

According to the dimensions of the scaffolds of this assay (1 cm high, 1 cm in diameter and a channel of 5 mm in diameter), a volume of 3141.59 mm³ was obtained. To this value, the volume of the inner channel must be subtracted, which has a value of 785.39 mm³. Therefore, the working volume is equal to 2356.2 mm³. This value is used to calculate the number of cells that this scaffold must carry by means of the following equation:

3D scaffold cells (1 cm high)=2356.2 mm³×(2.5×10⁴ cells/23.562 mm³)

Cells in 3D scaffold (1 cm high)=2,500,000 CEM-GW

For this pilot assay, scaffolds previously sterilized by Gamma radiation were used.

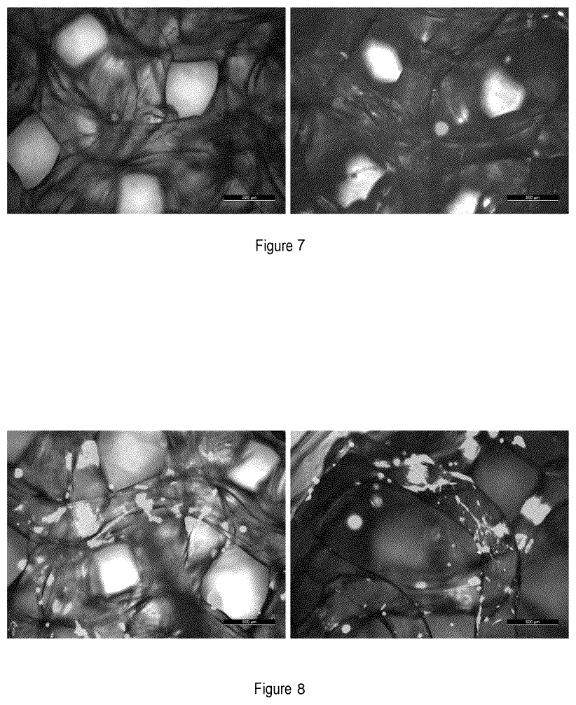

Since the scaffolds used are of lower height, for cell culture, a 200 μL cell suspension was prepared with 2×10⁶ CEM-GW and deposited in the internal channel of the scaffold. After 6 hours post-culture, 1 mL of supplemented culture medium is slowly added, taking care to avoid the detachment of the cells contained in the polymeric matrix. After 24 hours in incubator and with supplemented culture medium, the evaluation of cell viability was carried out through the live/dead kit. This kit has two reagents (calcein and ethidium homodimer) that show green-colored living cells (calcein) and red-colored dead cells (ethidium homodimer) when viewed with a fluorescence microscope. FIG. 7 shows the images collected by the fluorescence microscope corresponding to the live/dead staining in the assay described above.

One of the observations of this trial is oriented to the difficulty of focusing the cells on the scaffold. Due to the interior architecture it is not possible to show a completely sharp plan of both things. As for the viability assay (“live/dead”), little cell adhesion was found on the three-dimensional polymer matrix, being attributed to an overflow of the cell suspension towards the culture plate during the incubation prior to the addition of culture medium. Despite this, some cells were able to adhere, which showed to be mostly alive (approximately 95%). Additionally, with this assay it was possible to stipulate the critical steps for the cell seeding process in these structures, which allows to optimize and improve the viability evaluation process in these materials.

### Electrospinning

In order to improve cell adhesion on the polymeric matrix and prevent the invasion of the muscle tissue adjacent to the bone when it is implanted in animal models and subsequently in humans, a PLA/HA composite coating was incorporated. Said coating is generated by electrospinning 300 μL of polymeric solution directly on the 3D scaffolds located in the static collector, which generates that the fibers wrap the printed scaffolds. The concentration of the PLA/HA solution has a concentration of 10% w/v PLA and 1% w/v HA. The parameters used for electrospin coating are a voltage of 15 Kv, a distance between the needle tip and the collector of 15 cm with a flow rate of 1 ml/hr. This outer membrane is oriented to mimic the composition and functions of the periosteum, as well as contain the medium and the cells inside the scaffold, which guarantees the cell-scaffold interaction and allows to reduce the probability of an overflow of the cell suspension. The device obtained from this combination of 3D printing and electrospinning was called “3D Quyne”.

### In Vitro Assay of the “3D Quyne” Device

After coating the 3D scaffolds, the in vitro evaluation was carried out where the same seeding procedure described above was carried out. Briefly, a 200 μL cell suspension with 2×10⁶ CEM-GW was deposited on the internal channel of the scaffold. After 24 hours in incubator and with supplemented culture medium, the evaluation of cell viability was carried out through the live/dead kit. The images obtained from this assay are shown in FIG. 8.

According to the images of FIG. 8, a viability rate greater than 75% and a greater number of cells adhered to the interior of 3D Quyne compared to the uncoated scaffold is evidenced (see FIG. 7). In accordance with this, it can be indicated that the coating with the electrospun membrane retained the cell suspension inside the device, making it possible for the cells to adhere to the polymer.

### Quantification of Growth Factors

Another assay carried out in vitro consisted of the quantification of growth factors secreted by the GW-MECs seeded on said polymeric matrices through Luminex. For this, an electrospun PLA membrane and a 3D PLA scaffold were evaluated. The results obtained are shown in Table 2.

TABLE 2
Growth factors quantified on polymeric scaffolds.

| | Aggrecan | FGFb | VEGF | HGF |
|---|---|---|---|---|
| Scaffold | (pg/mL) | (pg/mL) | (pg/mL) | (pg/mL) |
| Electrospun PLA | 2919.326 | 277267 | 1467.871 | 18428.469 |
| PLA 3D | 3472.321 | 7012.277 | 6643.605 | 31784.728 |

With the values obtained after the Luminex assay, higher concentrations of the factors were found in the three-dimensional scaffold compared to the electrospun membrane. These factors are involved in the processes of bone repair, therefore, the three-dimensional structure contributes positively to the secretion of said compounds by the cells contained within it, which can accelerate the processes of bone repair.

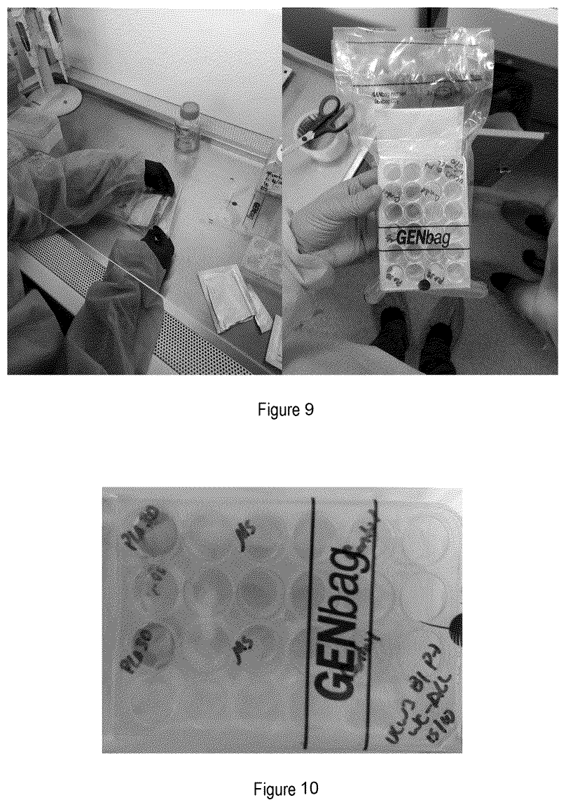

The results obtained provided the basis for scaling at the in vivo level, however, this entails evaluating new variables, including the transfer of the device with cells from the laboratory to the biosphere. That is why a study was carried out in which the cell survival in the scaffolds was estimated after subjecting them to room temperature and variable CO₂ atmosphere for 4 hours. To do this, the culture dish was placed inside a GENbag which was sealed with a CO₂ generator inside. FIG. 9 shows the arrangement of the culture plate in the GENbag with the CO₂ generator.

The time to which the scaffolds were subjected is greater than the estimated travel time, however, the time before being implanted in the biomodels was also taken into account, which is why an extended period of time was contemplated with these conditions. At the end of the evaluation time, the scaffolds presented changes in the coloration of the culture medium, as can be seen in FIG. 10.

The DMEM culture medium has a compound that allows elucidating the pH changes that may occur during cell culture. In the case of an increase in alkalinity, the culture medium tends to become intense pink, while an increase in acidity causes the medium to turn yellow. As observed, the acidity of the medium increases after being under the conditions evaluated, attributed to an increase in carbonic acid in the medium.

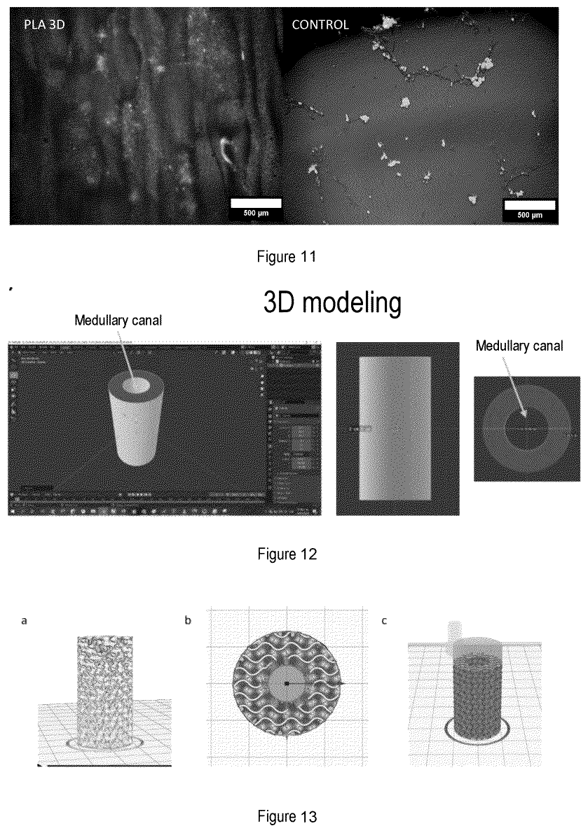

To observe changes in cell viability under these conditions (CO₂ and temperature), staining was performed with the live/dead kit with which live cells (green color) and dead cells (red color) on the scaffolds can be noticed. The images obtained after staining are shown in FIG. 11.

The low sharpness of these images is generated by the membrane that covers the three-dimensional structure, which restricts the passage of light. On the other hand, despite the pH change in the culture medium, a blurred green area was observed inside the scaffold that corresponds to living cells, with which a percentage of cell viability greater than 70% was determined. During the assay it was evident that the culture medium remains inside the scaffold, therefore, the presence of the cells inside the construct was ensured and the probability of cell adhesion was increased. Regarding the control cells in an adherent culture plate, a percentage of viability greater than 95% is observed. According to these results, the feasibility of employing the coated PLA 3D scaffolds for seeding CEM-GW at room temperature and with unstable CO₂ atmosphere for 4 hours was demonstrated.

### Example 2. Studies and Preclinical Evaluation of the Device Called 3D Quyne

The in vivo evaluation of 3D Quyne was carried out in lagomorphic biomodels with an age between 3 and 4 months and a weight between 2 Kg to 3 Kg approximately. Biomodels of these features have a femur of 1 cm in diameter and an average length of 8 cm, where 6 cm correspond to the diaphysis. According to what is reported in the literature, a critical bone injury corresponds to twice the diameter of the compromised bone tissue. These types of injuries have no natural repair, so grafts that fill this space are often implanted. In that sense, 3D Quyne was designed and modeled for a critical bone defect in rabbit corresponding to 2 cm long and 1 cm in diameter (see FIG. 12).

The file is then exported in STL format and imported into a Slic3r program to configure printing. In the software, the dimensions of the device are verified and the printing simulation is made, as shown in FIG. 13. After this, the printing process is carried out using the previously established parameters.

Subsequently, the device is taken to the laboratory where the coating is carried out with the electrospinning, as described in the in vitro assays. The obtained device is left in a 37° C. convection heating oven to remove traces of solvent and then Gamma radiation sterilization is performed.

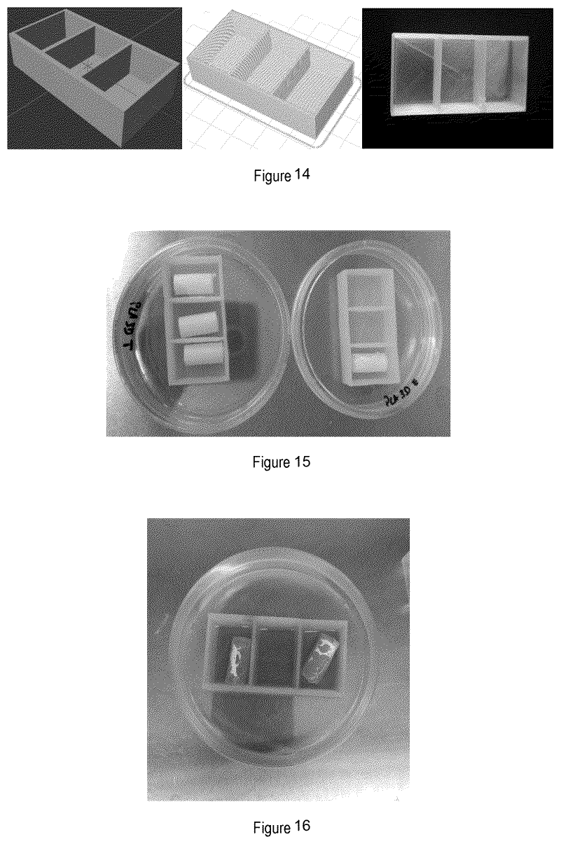

On the other hand, a container was designed and manufactured that would allow the 2 cm high 3D Quyne device to be available. The manufacturing process of the container is shown in FIG. 14.

The container has three spaces each 3 cm high, 2 cm wide and 1.5 cm deep. It was printed with a double wall to prevent the transfer of culture medium between wells and to the outside. The supports were manufactured in PLA, given the ease of printing and sterilization of the material in an autoclave. To evaluate the feasibility of using the container, a test was carried out with two 2 cm high scaffolds and the behavior of the material was observed under traditional cultivation conditions giving favorable results.

Prior to cell seeding on 3D Quyne, sterilization of the supports was carried out through an autoclave at 120° C. On the other hand, the scaffolds were irradiated with gamma rays. After both processes, the devices were deposited in the containers and placed in Petri dishes as shown in FIG. 15. It should be noted that this process was carried out inside the cabin to avoid possible contamination.

Cellular functionalization of the device was performed with a total of 8×10⁶ CEM-GW, which were seeded in two steps, each with a 250 μL cell suspension containing 4×10⁶ CEM-GW and 12 hours apart. This variation in cell culture methodology was performed in order to ensure uniform cell density throughout the device. After 12 hours from the last sowing, sufficient culture medium was added to cover the scaffolds completely, as shown in FIG. 16.

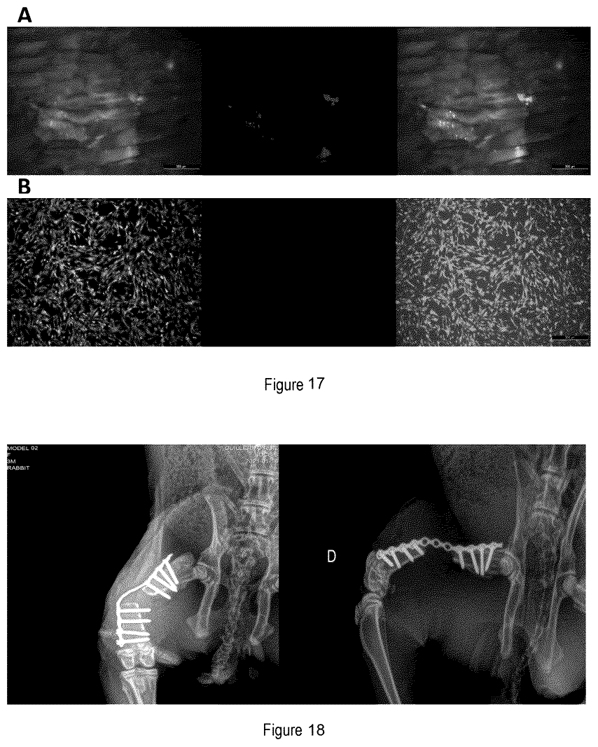

Like the previous assays, the cell viability test was performed with the live/dead kit, after subjecting the scaffolds to 4 hours at room temperature and under a variable CO₂ atmosphere. The images obtained are shown in FIG. 17.

The images obtained in this assay show a cell viability greater than 85% in both the device and the control plate after being subjected to these conditions. Some dead cells were also found in the external areas of the scaffold, while the living cells are located inside the device. In the same way as the previous assays, the visualization of the cells inside the scaffold is restricted by the architecture and the coating.

The seeding of CEM-GW was carried out three days prior to surgery. To evaluate the viability of the cells in the device, two controls were carried out, the first was transported to the bioterium and subsequently returned to the institute after surgery and the second control remained in the incubator of the IDCBIS laboratory.

In total, 6 osteotomies were performed, in the order described below:
1. Biomodel with electrospun PLA/COL/HA membrane.
2. Biomodel with electrospun PLA/COL/HA membrane.
3. Positive Control: Biomodel with autograft.
4. Biomodel with functionalized 3D Quyne device.
5. Biomodel with functionalized 3D Quyne device.
6. Negative Control: Biomodel with nothing.

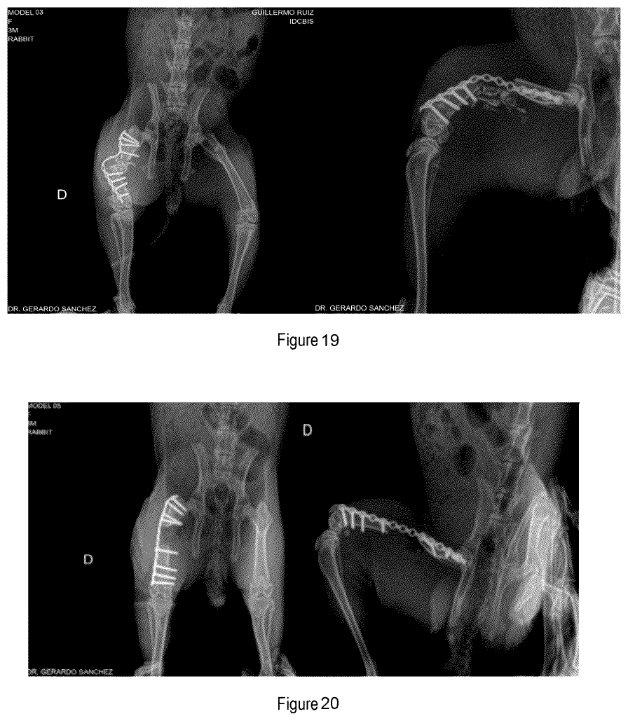

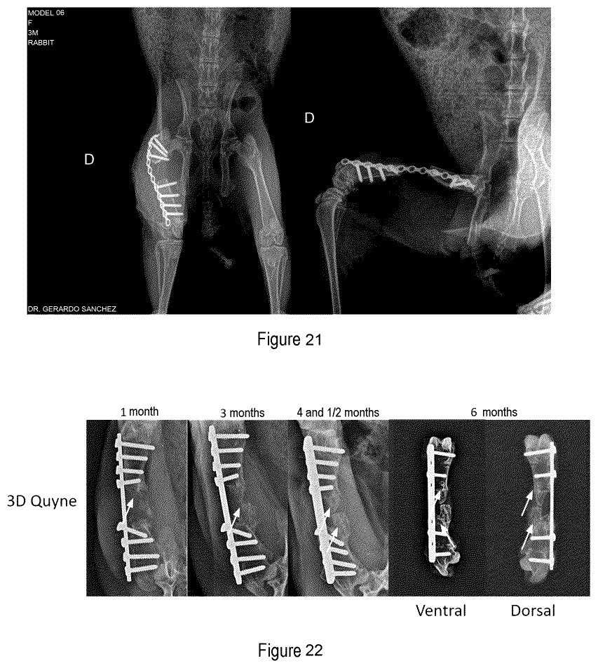

At 24 hours post-surgery, two radiographs are taken of each biomodel, one of the anteroposterior and one of the anterolateral area. These images allowed the integrity of the plates and screws used as fasteners to be observed (see FIGS. 18, 19, 20 and 21).

According to the radiographs, there are differences between each treatment, being the “3D Quyne” functionalized device the one that shows the best results in terms of the stability of the compromised bone tissue, since the device helps to support the force exerted by the limb when it was supported, contributing to maintaining the integrity of the fastening plate. In the case of the other treatments, the fastening plate must support the tissue on its own and withstand the force that the limb can exert, which increases the risk of limb failure and generates complications in the biomodels.

After 15 days, the biomodels were sacrificed due to the failure of the fastening plate in 5 of the 6 biomodels. Due to this, a new preclinical trial was carried out, but this time with four biomodels and a stainless steel fastening plate. In these biomodels, the 3D Quyne device was implanted with and without CEM-GW. This study served to assess the biocompatibility and contribution of the device in the repair of critical bone defects.

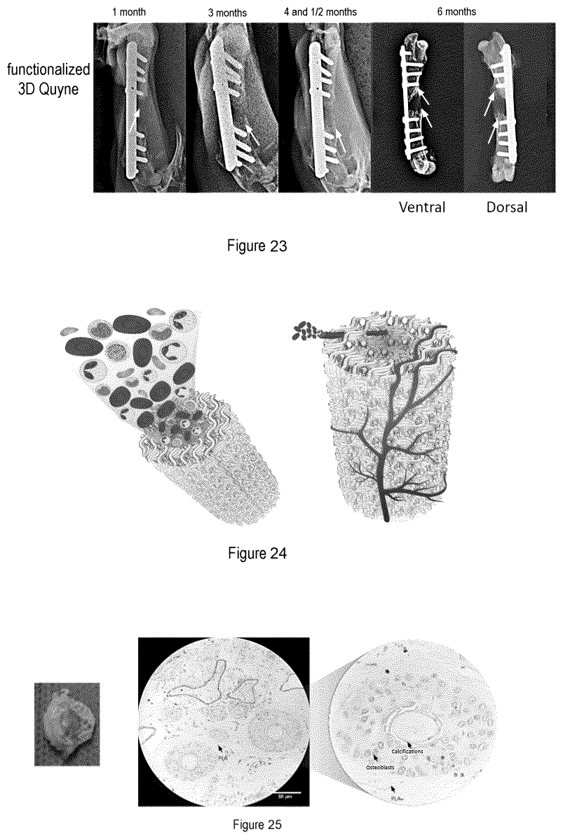

Both the pre-surgery preparation and the surgical process were performed as described above. The radiographs obtained in said study are shown in FIGS. 22 and 23.

Radiographic monitoring performed on the biomodels shows similar behavior in both treatments. Bone formation is found in all devices, therefore the 3D Quyne device with and without CEM-GW promotes the repair of critical bone injuries. In addition, a coating of the device composed of bone tissue is observed, which facilitates the communication of the compromised ends and helps the ossification process.

One of the factors that significantly influences bone repair is associated with tissue vascularization. The architecture of the 3D Quyne device promotes the processes of neovascularization through it. In addition, the internal channel has the exact conformation to communicate the bone ends and allow the transit of bone marrow and other cellular components involved in repair through the device (see FIG. 24). This generates an acceleration in the repair process, since the formation of blood vessels is a fundamental step in the repair.

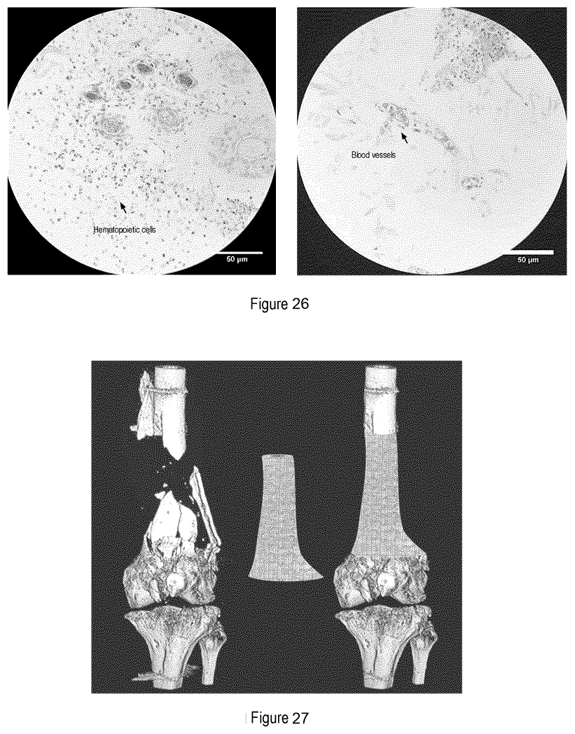

In the histological analysis of the bone samples obtained from the first preclinical assay, calcifications, blood vessels, osteoblasts and polymeric material are visualized. FIG. 25 shows the device removed from the injury site and a microscope visualization of the histology slides.

Likewise, in FIG. 26, possible blood vessel formations containing red and white blood cells can be seen within the 3D Quyne device. Likewise, possibly hematopoietic cells are visualized on the polymeric matrix. The design of this device is a good support for the proliferation of differentiated cells and the development of new bone tissue. For this reason, from these results, it is proposed to run more assays.

Another advantage offered by the 3D Quyne device lies in the possibility of modifying its shape and dimensions to adapt to different bone injuries. As an example, the design and modeling of the device adapted to a comminuted femur fracture is shown (see FIG. 27). There, the device can be implanted without generating additional trauma to the tissue since its features allow it to have a degradation rate according to bone turnover. Additionally, it will guide, promote the bone repair process and contribute to the mechanical support of the limb.

1. A method of manufacturing a device for repairing fractures or considerable bone injuries, wherein the method comprises the steps of:
designing from diagnostic images the device, wherein an area of interest is determined, the dimensions of the device and an interior channel of the device is designed;
3D printing the device layer by layer in polylactic acid, polycaprolactone, PLG, PLGA or polyalkylcyanoacrylates, wherein printing parameters selected from the group consisting of nozzle temperature, printing platform temperature, layer height, printing path width and printing speed are set, wherein the internal architecture of the device is realized by means of a gyroid infill pattern;
coating the device printed by electrospinning with a polymeric membrane obtained by means of a polymeric solution comprising between 7 and 20% of polylactic acid, between 1% and 5% of hydroxyapatite and 2,2,2 trifluoroethanol as solvent.

2. The method of claim 1, wherein the diagnostic images are selected from 3D radiographs and axial tomography.

3. The method of claim 1, wherein the temperature of the nozzle varies between 200° C. and 215° C., the temperature of the printing platform varies between 30° C. and 70° C., the height of the layer varies between 0.1 mm and 0.2 mm, the width of the printing path varies between 0.2 mm and 0.4 mm and the printing speed is 70 mm/s.

4. The method of claim 1, wherein the print density is 40% or the distance between lines varies between 0.8 mm and 1.1 mm.

5. The method of claim 1, wherein the 3D printing is performed on a fused deposition modeling machine.

6. The method of claim 1, wherein the solution from the electrospinning step is subjected to 37° C. and agitation ranging from 400 rpm to 600 rpm for at least 5 hours.

7. The method of claim 1, wherein the coated device is performed by electrospinning between 0.2 mL and 0.5 mL of the polymer solution directly onto the printed device, by manual rotation using a voltage between 10 kV and 18 kV, a distance between 10 cm and 18 cm, and an injection rate between 0.6 mL/h and 3 mL/h.

8. The method of claim 1, wherein said method further comprises a step of convection drying at a temperature of 37° C. for 48 hours.

9. The method of claim 1, wherein said method further comprises a gamma radiation sterilization step with a range ranging from 20 kGy to 25 kGy.

10. A medical device obtained by claim 1, wherein the device comprises a device composed of polylactic acid, polycaprolactone, PLG, PLGA or polyalkylcyanoacrylates, with an inner channel equivalent to half the total diameter of the bone and a coating comprising between 7% and 20% polylactic acid and between 1% and 5% hydroxyapatite, wherein further the device has a macroporous internal architecture ranging between 200 micrometers and 800 micrometers.

11. The medical device of claim 10, wherein the device can be functionalized with growth factors selected from the group consisting of VEGF, FGF, PDGF, BMP2, BMP4, BMP7, and cells of mesodermal origin.
